(19) United States
(12) Patent Application Publication
(10) Pub. No.: US 2018/0036219 A1
(43) Pub. Date: Feb. 8, 2018

(54) AEROSOL COMPOSITION FOR COLLECTING CARBON DIOXIDE

(71) Applicant: AMOREPACIFIC CORPORATION, Seoul (KR)

(72) Inventors: Ho Hyun SONG, Gyeonggi-do (KR); Jin Seob SHIN, Gyeonggi-do (KR); Joon Young HWANG, Gyeonggi-do (KR); Soon Ae AN, Gyeonggi-do (KR); Byung Young KANG, Gyeonggi-do (KR)

(73) Assignee: AMOREPACIFIC CORPORATION, Seoul (KR)

(21) Appl. No.: 15/553,899

(22) PCT Filed: Feb. 17, 2016
(86) PCT No.: PCT/KR2016/001585

§ 371 Date: Aug. 25, 2017

(30) Foreign Application Priority Data
Feb. 27, 2015 (KR) 10-2015-0027695

(51) Int. Cl. A61K 8/73; A61Q 19/00; A61K 8/34

(52) U.S. Cl. CPC A61K 8/73; A61K 8/34; A61Q 19/00

(57) ABSTRACT

The present invention relates to a composition for collection of carbon dioxide using a liquid surfactant. The aerosol composition containing a liquid surfactant, of the present invention, has no deterioration in quality even at high temperatures, absorbs carbon dioxide to form a predetermined shaped foam when sprayed on the skin, is smoothly absorbed when rolled several times, and is naturally absorbed into the skin after several minutes even when left by completely losing carbon dioxide, and thus the aerosol composition can be favorably used as a foam type cosmetic product.

### TECHNICAL FIELD

The present invention relates to, and more particularly, to a composition for capturing carbon dioxide using a liquid surfactant.

### BACKGROUND ART

An interface refers to a boundary surface at which gas and liquid, liquid and liquid, or liquid and solid meet each other. A surfactant is a compound having a hydrophilic portion which is easy to be dissolved in water, and a hydrophobic portion which is easy to be dissolved in oil. The surfactant serves to mitigate the boundary of the interface. Due to such property, it has been used a lot as soap or detergent. The molecules of the surfactant gather at a certain concentration or higher, and form a micelle structure. The micelle is formed when the surfactant has a concentration higher than a critical micelle concentration, and a temperature higher than a critical micelle temperature. When the micelle is formed in water, the hydrophobic portions of the surfactant gather at a center part to form a nucleus, and the hydrophilic portions form an outer part in contact with water. Like oil, the hydrophobic material is positioned in the inside of the micelle, thereby being stabilized, and dissolved in water, which is called, solubilization, and this is a basic principle of a detergent action.

The surfactant is used a lot as an emulsifier and a moisturizer of food or cosmetics, in addition to being used as detergent. The properties of the surfactant are greatly changed only by modifying some chemical structure, and also the kind of surfactant is various.

Generally, the surfactant may be classified into anionic, cationic, amphoteric and non-ionic surfactant, and the like, by the charge of the hydrophilic portion when dissociated in water.

The anionic surfactant may impart a charge to the interface of the outermost layer of a particle, thereby maximizing steric hindrance, and thus, preventing coalescence between particles to improve stability of the particles, so that a multiple emulsion phase form may be stably maintained even at low and high temperatures, and water-soluble and oil-soluble effective components may be stabilized to be effectively transferred. As the anionic surfactant, fatty acid soap (sodium laurate, sodium palmitate, etc.); higher alkyl sulfuric ester salts (sodium lauryl sulfate, potassium lauryl sulfate, etc.); alkyl ether sulfuric ester salts (polyoxyethylene (POE)-triethanolamine lauryl sulfate, POE-sodium lauryl sulfate, etc.); N-acyl sarcosinate (sodium lauroyl sarcosinate, etc.); higher fatty acid amide sulfonate (sodium N-myristoyl-N-methyl taurate, palm oil fatty acid sodium methyl taurate, sodium lauryl methyl taurate, etc.); phosphoric ester salts (sodium POE-oleyl ether phosphate, POE-stearyl ether phosphate, etc.); sulfosuccinates (sodium di-2-ethylhexyl sulfosuccinate, sodium monolauroyl monoethanolamide polyoxyethylene sulfosuccinate, sodium lauroyl polypropyleneglycol sulfosuccinate, etc.); alkylbenzene sulfonates (sodium linear dodecyl benzene sulfonate, triethanolamine linear dodecyl benzene sulfonate, linear dodecyl benzene sulfonate, etc.); higher fatty acid ester sulfuric ester salts (hardened palm oil fatty acid glycerin sodium sulfate, etc.); N-acyl glutamate (monosodium N-lauroyl glutamate, disodium N-stearoyl glutamate, monosodium N-myristoyl-L-glutamate or the like); sulfated oil (turkey red oil, etc.); POE-alkyl ether carboxylic acid; POE-alkyl allyl ether carboxylates; α-olefin sulfonate; higher fatty acid ester sulfonates; secondary alcohol sulfuric ester salts; higher fatty acid alkylolamide sulfuric ester salts; sodium lauroyl monoethanolamide succinate; N-palmitoyl asparaginate ditriethanolamine; sodium caseinate; or the like may be used.

As the example of the cationic surfactant, particularly selectively, salts of polyoxyalkylenated primary, secondary or tertiary fatty acid amine, and selectively a polyoxyalkylenated quaternary ammonium salt may be listed. Preferably, the cationic surfactants may be selected from polyoxyalkylated quaternary ammonium salts. As a preferred cationic surfactant, a quaternary ammonium salt, particularly behenyltrimethyl ammonium chloride, cetyltrimethyl ammonium chloride, quaternium-83, quaternium-87, behenylamidopropyl-2,3-dihydroxypropyl-dimethylammonium chloride, palmitylamidopropyl trimethylammonium chloride, stearamidopropyl-dimethyl-amine and the like; and also an alkyltrimethylammonium salt (stearyltrimethylammonium chloride, lauryltrimethylammonium chloride, etc.); an alkylpyridinium salt (cetylpyridinium chloride etc.); distearyl dimethyl ammonium dialkyl dimethyl ammonium chloride; poly(N,N-dimethyl-3,5-methylenepiperidinium) chloride; an alkyl quaternary ammonium salt; alkyldimethylbenzyl ammonium salts; an alkylisoquinolinium salt; a dialkylmorphonium salt; POE-alkylamine; an alkylamine salt; a polyamine fatty acid derivative; an amylalcohol fatty acid derivative; benzalkonium chloride; or benzethonium chloride is used.

The amphoteric or zwitterionic surfactant may be particularly an imidazoline-based amphoteric surfactant (sodium 2-undecyl-N,N,N-(hydroxyethylcarboxymethyl)-2-imidazoline, 2-cocoyl-2-imidazolinium hydroxide-1-carboxyethyloxy disodium salt, etc.); a betaine-based surfactant (2-heptadecyl-N-carboxymethyl-N-hydroxyethylimidazolinium betaine, lauryldimethylaminoacetic acid betaine, alkylbetaine, amidebetaine, sulfobetaine, etc.) or the like.

The non-ionic surfactant is generally obtained by adding alkylene oxide such as ethylene oxide (EO) and propylene oxide (PO) to a material having active hydrogen such as alcohol, and has properties such as a low critical micelle concentration, high solubilization ability, surface tension at low concentration and low bubbling ability as compared with ionic surfactants. Thus, it has a wide range of applications such as a fiber processing agent, detergent, an emulsifier and a dispersant, and is being used in various fields. Further, the non-ionic surfactant is not ionized in an aqueous solution, and has a hydroxide functional group and an ether functional group bond in the molecule, so that a lipophilic group becomes water-soluble, and thus, most of the non-ionic surfactants are water-soluble surfactants of which the molecule itself has surface activity.

Cosmetics may be generally classified into skincare products, make-up cosmetics, hair cosmetics, aromatic cosmetics, body cosmetics and the like, and depending on the formulation, may be classified into solubilized products, emulsified products, dispersed products and the like. The skincare products are classified into skin lotion having very low viscosity, mist having liquid viscosity, lotion having medium viscosity, essence having high viscosity, and cream having very high viscosity, depending on the viscosity of the basic cosmetics, and have been manufactured in various forms, depending on the use and purpose of the cosmetics.

Recently, as the boundary between a lotion formulation and essence gradually disappeared, preference of the lotion formulation has been decreased. For differentiated feeling of using the lotion formulation, an emulsion having low viscosity has been developed, however, when the emulsion has generally low viscosity, it is easily separated into an oil phase and an aqueous phase, which leads to an unstable formulation. Thus, various novel emulsification systems for overcoming this disadvantage are being studied. In recent years, for improving the feeling of using the formulation, the need to develop the cosmetics in an aerosol form having low viscosity has arisen. When developing the cosmetics in the aerosol form, various surfactants may be used, however, in this case, a long term stability problem of the formulation and the like may occur, and in the case that aerosol foam is formed in a large shape, it is similar to a general cleaning agent, thereby having unpleasant feeling of use, and in the case of being formed in a small shape, the preparation of an aerosol allowing suitable foam to be formed is needed.

Korean Patent Publication No. 10-1063333 discloses a hypoallergenic cleaning agent composition, however, by using non-ionic, anionic and amphoteric surfactants together, the use may be limited to those people with skin diseases such as atopy or acne. Further, Korean Patent Publication No. 10-1125922 discloses a cosmetic composition having low viscosity, however, the composition only improves emulsification stability by mixing a non-ionic surfactant and a glyceryl ester-based surfactant, and the use thereof may be limited to those people who are sensitive to skin irritation.

Accordingly, the development of aerosol which solves the above problems, has pleasant feeling of use even with the use of a sticky non-ionic surfactant, and has an excellent foam formulation is desperately needed.

### DISCLOSURE

### Technical Problem

An object of the present invention is to provide an aerosol composition maintaining quality depending on temperature, forming a suitable foam form when sprayed on the skin, and showing an effect of excellent feeling of use, by using a liquid surfactant.

### Technical Solution

In one general aspect, an aerosol composition for capturing carbon dioxide is prepared by including: carrying out mixing so that 1-5 wt % of a liquid surfactant, 1-15 wt % of polyol, 1-3 wt % of a thickener, 0.01-1.00 wt % of a preservative, 0.01-1.00 wt % of a flavoring agent, and a residual amount of purified water are included, based on the total weight of the composition.

The liquid surfactant herein is not limited, but may include any one or two or more selected from the group consisting of PEG-20 glyceryl isostearate, polysorbate 20, and polyglyceryl-2 diisostearate.

The polyol herein is not limited, but may include any one or two or more selected from the group consisting of glycerin, ethylene glycol, propylene glycol, butylene glycol, pentylene glycol, hexylene glycol, 1,2-hexanediol, octanediol, sorbitol, and polyethylene glycol.

The thickener herein is not limited, but may include any one or more selected from the group consisting of methyl cellulose, ethyl cellulose, hydroxymethyl cellulose, hydroxyethyl cellulose, Carbopol 941, Carbopol 934, Carbopol 940, Aqupec HV-505E, Aqupec HV-501, HV-504, polyacrylic acid, polymethacrylic acid, polyacrylate 13/polyisobutene/polysorbate, acrylate/C10-30 alkyl acrylate copolymer, Carbopol ETD2020, guar gum, carrageenan gum, xanthan gum, and ammoniumacryloyldimethyltaurate vinylpyrrolidone copolymer.

In another general aspect, a method of preparing an aerosol composition for capturing carbon dioxide includes carrying out mixing so that 1-5 wt % of a liquid surfactant, 1-15 wt % of polyol, 1-3 wt % of a thickener, 0.01-1.00 wt % of a preservative, 0.01-1.00 wt % of a flavoring agent, and a residual amount of purified water are included, based on the total weight of the composition.

The liquid surfactant herein is not limited, but may include any one or two or more selected from the group consisting of PEG-20 glyceryl isostearate, polysorbate 20, and polyglyceryl-2 diisostearate.

The polyol herein is not limited, but may include any one or two or more selected from the group consisting of glycerin, ethylene glycol, propylene glycol, butylene glycol, pentylene glycol, hexylene glycol, 1,2-hexanediol, octanediol, sorbitol, and polyethylene glycol.

The thickener herein is not limited, but may include any one or more selected from the group consisting of methyl cellulose, ethyl cellulose, hydroxymethyl cellulose, hydroxyethyl cellulose, Carbopol 941, Carbopol 934, Carbopol 940, Aqupec HV-505E, Aqupec HV-501, HV-504, polyacrylic acid, polymethacrylic acid, polyacrylate 13/polyisobutene/polysorbate, acrylate/C10-30 alkyl acrylate copolymer, Carbopol ETD2020, guar gum, carrageenan gum, xanthan gum, and ammoniumacryloyldimethyltaurate vinylpyrrolidone copolymer.

In another general aspect, cosmetics are prepared by the above preparation method.

### Advantageous Effects

The aerosol composition including the liquid surfactant of the present invention is characterized in that it does not undergo deterioration of quality even at high temperature, absorbs carbon dioxide when sprayed on the skin to form a certain form of foam, is softly absorbed into the skin when rolled thereon several times, and naturally loses carbon dioxide completely after several minutes even when left as it is, thereby being absorbed into the skin. Thus, it is expected to be usefully applied as cosmetics in a foam form.

### BEST MODE

The present invention relates to an aerosol composition for capturing carbon dioxide prepared by including: carrying out mixing so that 1-5 wt % of a liquid surfactant, 1-15 wt % of polyol, 1-3 wt % of a thickener, 0.01-1.00 wt % of a preservative, 0.01-1.00 wt % of a flavoring agent, and a residual amount of purified water are included, based on the total weight of the composition.

The liquid surfactant herein is not limited, but may include any one or two or more selected from the group consisting of PEG-20 glyceryl isostearate, polysorbate 20, and polyglyceryl-2 diisostearate.

The surfactant herein is not limited, but preferably, may include a lipophilic surfactant, a hydrophilic surfactant, or both of them.

The lipophilic surfactant is not limited, but one or more selected from the group consisting of polyglyceryl-3-dipolyhydroxystearate, polyoxyethylene(30)-dipolyhydroxystearate, polysiloxane polyalkyl polyether copolymer (ABIL EM-90), cyclomethicone/dimeticone copolymer, polyglyceryl-3-diisostearate, polyglyceryl-2-tetraisostearate, polyglyceryl-2-triisostearate and polyglyceryl-4-isostearate may be used.

The hydrophilic surfactant is not limited, but one or more selected from the group consisting of polyoxyethylene(10)-hydrogenated castor oil, polyoxyethylene(40)-hydrogenated castor oil, polyoxyethylene(60)-hydrogenated castor oil, polysorbate 60, polysorbate 80 and polysorbate 20 may be used.

The non-ionic surfactant which is generally obtained by adding alkylene oxide such as ethylene oxide (EO) and propylene oxide (PO) to a material having active hydrogen such as alcohol, has properties such as a low critical micelle concentration, high solubilization ability, surface tension at low concentration and low bubbling ability, as compared with ionic surfactants, and thus, has a wide range of applications such as fiber processing agents, detergents and emulsifiers, and is being used in various fields. Further, the non-ionic surfactant is not ionized in an aqueous solution, and has a hydroxide functional group and an ether functional group bond in the molecule, so that a lipophilic group becomes water-soluble, and thus, most of the non-ionic surfactants are water-soluble surfactants of which the molecule itself has surface activity. In the non-ionic surfactant, the hydrophilic group does not consist of an ionic group, and is partially charged, and thus, the non-ionic surfactant improves the stability of the emulsion like other ionic surfactants. However, it generally has low irritation as compared with the ionic surfactant, and thus, is often used in products close to a human body like a cosmetic material.

It is preferred that the content of the non-ionic surfactant is 1-5 wt %, based on the total weight of the composition. This is because when the content of the non-ionic surfactant is too low, which is less than 1 wt %, the water-in-oil dispersion formulation is unstable, thereby being separated into an internal phase and an external phase during storage for several months, and when the content is too high, which is more than 5 wt %, a skin irritation problem is caused due to the surfactant.

It is possible for polyglyceryl-2-triisostearate according to an exemplary embodiment of the present invention which is a non-ionic surfactant having three alkyl chains on a polyglyceryl chain which is a hydrophilic group to produce a surface active material covered with an amorphous surfactant.

As the non-ionic surfactant, a lipophilic non-ionic surfactant or a hydrophilic non-ionic surfactant may be used, and it is not particularly limited as long as it may be used in a common cosmetic composition. For example, as the lipophilic non-ionic surfactant, sorbitan fatty acid esters (sorbitan monooleate, sorbitan monoisostearate, sorbitan monolaurate, sorbitan monopalmitate, sorbitan monostearate, sorbitan sesquioleate, sorbitan trioleate, penta-2-ethylhexyl acid diglycerol sorbitan, tetra-2-ethylhexyl acid diglycerol sorbitan, etc.); glycerinpolyglycerin fatty acids (monocottonseed oil fatty acid glycerin, glycerin monoerucate, glycerin sesquioleate, glycerin monostearate, glycerin α,α′-oleate pyroglutamate, glycerin malate monostearate, etc.); propylene glycol fatty acid esters (propylene glycol monostearate, etc.); a hardened castor oil derivative; glycerin alkyl ether, and the like may be used, and as the hydrophilic non-ionic surfactant, for example, one or more of POE (polyoxyethylene)-sorbitan fatty acid esters (POE-sorbitan monooleate, POE-sorbitan monostearate, POE-sorbitan tetraoleate, etc.); POE-sorbit fatty acid esters (POE-sorbit monolaurate, POE-sorbit monooleate, POE-sorbit pentaoleate, POE-sorbit monostearate, etc.); POE-glycerin fatty acid esters (POE-glycerin monostearate, POE-glycerin monoisostearate, POE-glycerin triisostearate, etc.); POE-fatty acid esters (POE-distearate, POE-monodioleate, ethylene glycol distearate, etc.); POE-alkyl ethers (POE-lauryl ether, POE-oleyl ether, POE-stearyl ether, POE-behenyl ether, POE-2-octyl dodecyl ether, POE-cholestanol ether, etc.); a Pluronic type (Pluronic, etc.); POE.POP (polyoxyethylene.Polyoxypropylene)-alkylethers (POE.POP-cetyl ether, POE.POP-2-decyl tetradecyl ether, POE.POP-monobutyl ether, POE.POP-hydrogenated lanoline, POE.POP-glycerin ether, etc.); tetra POE.tetra POP-ethylene diamine condensates (Tetronic, etc.); a POE-castor oil hardened castor oil derivative (POE-castor oil, POE-hardened castor oil, POE-hardened castor oil monoisostearate, POE-hardened castor oil triisostearate, POE-hardened castor oil monopyroglutamic monoisostearic diester, POE-hardened castor oil maleic acid, etc.); a POE beeswax.lanolin derivative (POE-sorbit beeswax, etc.); alkanol amide (palm oil fatty acid diethanol amide, lauric monoethanolamide, fatty acid isopropanolamide, etc.); POE-propylene glycol fatty acid ester; POE-alkyl amine; POE-fatty acid amide; saccharose fatty acid ester; alkylethoxydimethylamineoxide; trioleyl phosphoric acid may be used, and in the present invention, only those in a liquid state were used, with the exclusion of those in a solid state, but not limited thereto.

As a preferred surfactants herein, polyoxyethylene cetyl ether, polyoxyethylene oleyl ether, polyoxyethylene stearyl ether, polyoxyethylene nonylphenyl ether, polyoxyethylene lauryl ether, polyoxyethylene hexyldecyl ether, polyoxyethylene isostearyl ether, polyoxyethylene octyldodecyl ether, polyoxyethylene behenyl ether, polyoxyethylene cholesteryl ether, polyoxyethylene hardened castor oil, sorbitan ester, monofatty acid glycerin, trifatty acid glycerin, polyglycerin fatty acid ester, isostearic acid polyoxyethylene glycerin, triisostearic acid polyoxyethylene glycerin, monostearic acid polyoxyethylene glycerin, distearic acid polyoxyethylene glyceryl, tristearic acid polyoxyethylene glyceryl or the like may be used, but not limited thereto.

A moisturizer prevents freezing of a continuous phase at low temperature, is used for aiding in a moisturizing effect, and within a range of not interfering in the purpose of the present invention, water-soluble components, for example, glycol such as propylene glycol, 1,3-butylene glycol and dipropylene glycol, polyethylene glycol; glycerol such as glycerin, diglycerin and polyglycerin; sugars such as sorbitol, multitol, saccharose, starch sugar and lactitol; hyaluronic acid; betaine may be further used, but not limited thereto. This moisturizer increases the moisturizing ability of the skin, and decreases the polarity of a water-soluble phase, so that wax, higher alcohol, a surfactant and the like having low polarity may interact with the water phase to aid in forming a stable emulsion.

Further, the composition of the present invention may include 1.0 to 15 wt % of polyol, in order to provide the skin with moisturizing ability. The polyol is not limited, but for example, glycerin, ethylene glycol, propylene glycol, butylene glycol, pentylene glycol, hexylene glycol, 1,2-hexanediol, octanediol, sorbitol, polyethylene glycol, or the like may be used. Here, when the polyol is used at less than 1.0 wt %, the function to be provided for the composition may be unsatisfactory, due to the unduly low content. Further, by way of example, when the polyol is combined with glycerin and butylene glycol, reduced irritation and an antiseptic effect may be imparted, thereby using a combination of two components.

The thickener performs a function of adjusting the viscosity of a cosmetic material, and complementing stability due to the fluidity of a multiemulsion phase in which various water-soluble and oil-soluble effective components are stabilized. The thickener used herein is not limited, but for example, any one or more selected from the group consisting of methyl cellulose, ethyl cellulose, hydroxymethyl cellulose, hydroxyethyl cellulose, Carbopol 941, Carbopol 934, Carbopol 940, Aqupec HV-505E, Aqupec HV-501, HV-504, polyacrylic acid, polymethacrylic acid, polyacrylate 13/polyisobutene/polysorbate, acrylate/C10-30 alkyl acrylate copolymer, Carbopol ETD2020, guar gum, carrageenan gum, xanthan gum, and ammoniumacryloyldimethyltaurate vinylpyrrolidone copolymer may be used.

It is preferred that thickener may be included at 0.01 to 5 wt %, based on the total weight of the composition, in terms of maintaining the viscosity of a cosmetic material appropriately. Further, by way of example, it is preferred that an ammonium acryloyl dimethyllaurate vinyl pyrrolidone copolymer is used as the thickener, since an effect of thickening well is shown even under the environment of a lot of salts.

The kind of the oil used herein is not particularly limited, but a common oil used in a cosmetic composition may be used. For example, a hydrocarbon-based oil such as squalan and a mineral oil; an ester-based oil such as caprilic/capric triglyceride, neopentyl glycol dicaprate, 2-octyldodecylmyristate, isopropylmyristate, isocetylethylhexanoate, pentaerythrityl tetraethyl hexanoate, butylene glycol dicaprylate caprate, hexyl laurate, distearyl malate, cetyl 2-ethylhexanoate, octyl dodecanol and glyceryl triethyl hexanoate; a vegetable oil such as olive oil, avocado oil, jojoba oil and macadamia oil; an animal oil such as lanolin; or a silicon oil such as dimethylpolysiloxane, methylphenylpolysiloxane, decamethylcyclopentasiloxane, methyltrimethicone, phenyltrimethicone, cyclomethicone and dimethicone may be used alone or in combination. The oil may act as a factor affecting the use and stability of an emulsion and the like, depending on the selection and content of oily components, such as, dissolving solid components such as wax and higher alcohol, and affecting adhesion and spreading of an emulsion and the like.

The preservative herein is not limited, but any one or more selected from the group consisting of 3-hexyloxy-1,2-propanediol, 1,2-hexanediol, oxtanediol, paraben and phenoxyethanol may be used, and preferably phenoxyethanol may be used.

The composition of the present invention may further include higher alcohol, wax, a flavoring agent, a coloring agent and the like within the range of not interfering in the purpose of the present invention, and those components may be selected from those common in the art to which the present invention belongs.

The higher alcohol is a solid component having a great influence on the viscosity of a cosmetic composition, and for example, a straight chain alcohol (lauryl alcohol, cetyl alcohol, stearyl alcohol, behenyl alcohol, myristyl alcohol, oleyl alcohol, cetostearyl alcohol or the like); a branched chain alcohol (monostearylglycerin ether (batyl alcohol), 2-decyltetradecinol, lanolin alcohol, cholesterol, phytosterol, hexyldodecanol, isostearylalcohol, octyldodecanol, etc.) or the like may be used. The higher alcohol improves the stability of a formulation by increasing the viscosity of the emulsion to inhibit the association of oil particles.

The wax is a solid component maintaining the appearance of a water-in-oil type or oil-in-water type, and as the wax, candelilla wax, ceresin, carnauba wax, paraffin, vegetable wax, spermaceti, beeswax, cotton wax, very berry wax, white wax, montane wax, rice bran wax, lanolin, sugarcane wax, jojoba wax, microcrystalline wax or the like may be used. The wax provides gloss, and forms a protection film when applied to the skin, thereby protecting the skin and imparting heavy feeling of use.

Further, the present invention relates to a method of preparing an aerosol composition for capturing carbon dioxide including: carrying out mixing so that 1-5 wt % of a liquid surfactant, 1-15 wt % of polyol, 1-3 wt % of a thickener, 0.01-1.00 wt % of a preservative, 0.01-1.00 wt % of a flavoring agent, and a residual amount of purified water are included, based on the total weight of the composition.

Further, the present invention relates to cosmetics prepared by the above method.

Hereinafter, the present invention will be described in detail through the following examples. However, the following example is only to assist in the understanding of the present invention, and the scope of the present invention is not limited thereto in any sense.

### Example 1

2.00 g of liquid polyglyceryl-2 diisostearate (C42H82O7; COSMOL42V, The Nisshin OilliO, Ltd., Japan), 5.00 g of butylene glycol, 5.00 g of glycerol, 2.00 g of ammonium acryloyldimethyltaurate/vinylpyrrolidone copolymer (ARISTOFLEX AVC, Clariant, Switzerland), 0.30 g of phenoxyethanol, and 0.10 g of fragrance were mixed with 85.60 g of purified water, thereby preparing 100.00 g of an aerosol composition.

### Example 2

2.00 g of liquid polyethyleneglycol-20 glyceryl isostearate (EMALEX GWIS 120, NIHON EMULSION CO., LTD., Japan), 5.00 g of butylene glycol, 5.00 g of glycerol, 2.00 g of ammonium acryloyldimethyltaurate/vinylpyrrolidone copolymer, 0.30 g of phenoxyethanol, and 0.10 g of fragrance were mixed with 85.60 g of purified water, thereby preparing 100.00 g of an aerosol composition.

### Example 3

2.00 g of liquid polysorbate-20 (C₅₉H₁₁₄O₂₆; Alkest TW 20 TWEEN 20-LQ-(SG), CRODA, United Kingdom), 5.00 g of butylene glycol, 5.00 g of glycerol, 2.00 g of ammonium acryloyldimethyltaurate/vinylpyrrolidone copolymer, 0.30 g of phenoxyethanol, and 0.10 g of fragrance were mixed with 85.60 g of purified water, thereby preparing 100.00 g of an aerosol composition.

### Comparative Example 1

An aerosol composition was prepared in the same condition with the same components as in Example 1, except that 2.00 g of solid polyglyceryl-3 methylglucose distearate (TEGO CARE 450, EVONIK, Germany) was added instead of polyglyceryl-2 diisostearate.

### Comparative Example 2

An aerosol composition was prepared in the same condition with the same components as in Example 1, except that 2.00 g of solid C14-22 alcohol and C12-20 alkyl glucoside (a mixing ratio of C14-22 alcohol:C12-20 alkyl glucoside=20:80; MONTANOV L, SEPPIC, France) was added instead of liquid polyglyceryl-2 diisostearate.

### Comparative Example 3

An aerosol composition was prepared in the same condition with the same components as in Example 1, except that 2.00 g of solid polyethyleneglycol-40 stearate (C₂₀H₄₀O₃; MYRJ #52 S, CRODA, United Kingdom) was added instead of liquid polyglyceryl-2 diisostearate.

### Experimental Example 1

The aerosol composition prepared as described above was maintained at room temperature (25° C.), 37° C., 45° C. and 60° C., respectively, as described below, and then a 300 mL container equipped with a nozzle for spraying was filled with the composition, respectively, which was stored at room temperature for 3 weeks, and the state of the nozzle was observed. The results are shown in Table 1 below.

TABLE 1

| State of | | | | Comparative | Comparative | Comparative |
|---|---|---|---|---|---|---|
| nozzle | Example 1 | Example 2 | Example 3 | Example 1 | Example 2 | Example 3 |
| Room | Good | Good | Good | Good | Good | Good |
| temperature | | | | | | |
| (25° C.) | | | | | | |
| 37° C. | Good | Good | Good | Good | Good | Good |
| 45° C. | Good | Good | Good | Nozzle was | Nozzle was | Nozzle was |
| | | | | clogged | clogged | clogged |
| | | | | after 3 | after 3 | after 3 |
| | | | | weeks | weeks | weeks |
| 60° C. | Good | Good | Good | Nozzle was | Nozzle was | Nozzle was |
| | | | | clogged | clogged | clogged |
| | | | | after 1 week | after 1 week | after 1 week |

As shown in the results above, in the case of the present invention using the liquid surfactant, after raising the temperature of the aerosol composition to 60° C., and filling the container therewith, nozzle clogging did not occur even during long term storage. However, in the case of the control using the solid surfactant, after raising the temperature of the composition to 45° C. or more, and filling the container therewith, all nozzles were clogged in 1 to 3 weeks. It was confirmed therefrom that the aerosol composition using the solid surfactant underwent very large quality degradation when exposed to high temperature, and thus, marketability and convenience of use thereof was very poor.

### Experimental Example 2

As shown in the results above, the liquid aerosol composition hardly underwent quality degradation at general contact temperature, and thus, the shape and stickiness degrees of the foam which was formed by absorbing carbon dioxide from spraying according to the used liquid surfactant were measured, respectively.

FIG. 1 is a photograph of the foam having a diameter of 1 cm, formed by spraying the composition on the skin.

The results of measuring the height and stickiness degree of the foam formed for each composition are shown in Table 2.

TABLE 2

| | Example 1 | Example 2 | Example 3 |
|---|---|---|---|
| Foam height (cm) | 0.3-0.8 | 1.0-1.5 | 1.2-1.7 |
| Stickiness after absorption ^{a} | + | +++ | +++ |

^{a} Stickiness degree evaluation scale: +++ very sticky, ++ sticky, + no stickiness

As described above, it was confirmed that in the case that liquid polyglyceryl-2 diisostearate was used as the surfactant, the height of the foam was appropriately formed, and in the case of Examples 2 and 3, surface active force was too strong, so that foam in an artificial shape was formed, in which the height of the formed foam was similar to that of a general cleaning agent. Further, as a result of evaluating the stickiness degree, it was confirmed that in the case of using the liquid polyglyceryl-2-diisostearate, there was little stickiness, as compared with Examples 2 and 3, and when rolled on the skin 4-5 times, the foam softly collapsed and was absorbed into the skin in a liquid form. Further, when forming foam by spraying at a diameter of 1 cm, the number of bubbles in the foam having a size of 1 mm or more was 10 or less which is small, and most of the bubbles took a microbubble form in a micrometer scale. As such, most of the bubbles took a microbubble form, thereby greatly improving the feeling of use by the contact with the skin.

It was confirmed that the foam had a semi-spherical shape initially by capturing carbon dioxide, however, when left as it is, lost its form within 2 minutes, and completely lost carbon dioxide within 5 minutes, thereby being absorbed into the skin.

Through an exemplary embodiment of the present invention, an aerosol composition for capturing carbon dioxide including a liquid surfactant was prepared, thereby greatly improving the long term stability of the formulation and the feeling of using the aerosol, which were the conventional problems, and thus, the present invention was completed.

1. An aerosol composition for capturing carbon dioxide, comprising: a liquid surfactant, polyol, a thickener, a preservative, a flavoring agent, and purified water.

2. The composition of claim 1, wherein the liquid surfactant includes any one or two or more selected from the group consisting of PEG-20 glyceryl isostearate, polysorbate 20, and polyglyceryl-2 diisostearate.

3. The composition of claim 1, wherein the polyol includes any one or two or more selected from the group consisting of glycerin, ethylene glycol, propylene glycol, butylene glycol, pentylene glycol, hexylene glycol, 1,2-hexanediol, octanediol, sorbitol, and polyethylene glycol.

4. The composition of claim 1, wherein the thickener includes any one or more selected from the group consisting of methyl cellulose, ethyl cellulose, hydroxymethyl cellulose, hydroxyethyl cellulose, Carbopol 941, Carbopol 934, Carbopol 940, Aqupec HV-505E, Aqupec HV-501, Aqupec HV-504, polyacrylic acid, polymethacrylic acid, polyacrylate 13/polyisobutene/polysorbate, acrylate/C10-30 alkyl acrylate copolymer, Carbopol ETD2020, guar gum, carrageenan gum, xanthan gum, and ammoniumacryloyldimethyltaurate vinylpyrrolidone copolymer.

5. The composition of claim 1, wherein the liquid surfactant is comprised at 1-5 wt %, the polyol is comprised at 1-15 wt %, the thickener is comprised at 1-3 wt %, the preservative is comprised at 0.01-1.00 wt %, the flavoring agent is comprised at 0.01-1.00 wt %, and the purified water is comprised in a residual amount, based on a total weight of the composition.

6. A method of preparing an aerosol composition for capturing carbon dioxide, comprising: carrying out mixing so that 1-5 wt % of a liquid surfactant, 1-15 wt % of polyol, 1-3 wt % of a thickener, 0.01-1.00 wt % of a preservative, 0.01-1.00 wt % of a flavoring agent, and a residual amount of purified water are included, based on a total weight of the composition.

7. The method of claim 6, wherein the liquid surfactant includes any one or two or more selected from the group consisting of PEG-20 glyceryl isostearate, polysorbate 20, and polyglyceryl-2 diisostearate.

8. The method of claim 6, wherein the polyol includes any one or two or more selected from the group consisting of glycerin, ethylene glycol, propylene glycol, butylene glycol, pentylene glycol, hexylene glycol, 1,2-hexanediol, octanediol, sorbitol, and polyethylene glycol.

9. The method of claim 6, wherein the thickener includes any one or more selected from the group consisting of methyl cellulose, ethyl cellulose, hydroxymethyl cellulose, hydroxyethyl cellulose, Carbopol 941, Carbopol 934, Carbopol 940, Aqupec HV-505E, Aqupec HV-501, Aqupec HV-504, polyacrylic acid, polymethacrylic acid, polyacrylate 13/polyisobutene/polysorbate, acrylate/C10-30 alkyl acrylate copolymer, Carbopol ETD2020, guar gum, carrageenan gum, xanthan gum, and ammoniumacryloyldimethyltaurate vinylpyrrolidone copolymer.

10. Cosmetics prepared by the method of claim 6.

11. Cosmetics prepared by the method of claim 7.

12. Cosmetics prepared by the method of claim 8.

13. Cosmetics prepared by the method of claim 9.
